(19) United States
(12) Patent Application Publication
(10) Pub. No.: US 2026/0207904 A1
(43) Pub. Date: Jul. 23, 2026

(54) MAGNETICALLY CONTROLLED VIBRATING DRUG DELIVERY CAPSULE ROBOT AND SYSTEM

(71) Applicant: HUAZHONG UNIVERSITY OF SCIENCE AND TECHNOLOGY, Hubei (CN)

(72) Inventors: Quanliang CAO, Hubei (CN); Yue Ma, Hubei (CN); Feihong Xiang, Hubei (CN); Xiaotao Han, Hubei (CN); Liang LI, Hubei (CN)

(73) Assignee: HUAZHONG UNIVERSITY OF SCIENCE AND TECHNOLOGY, Hubei (CN)

(21) Appl. No.: 19/455,875

(22) Filed: Jan. 22, 2026

(30) Foreign Application Priority Data
Jan. 23, 2025 (CN) 202510108161.0

(51) Int. Cl. A61M 31/00; A61M 37/00

(52) U.S. Cl. CPC A61M 31/002; A61M 37/0092; A61M 2205/3515

(57) ABSTRACT

A magnetically controlled vibrating drug delivery capsule robot includes a power supply, a motor, a valve magnet and a limit magnet provided internally. In a default state, an attractive force exists between the valve magnet and the limit magnet, and under an effect of the attractive force, the valve magnet blocks an outlet of a drug storage compartment. Under an effect of an external relatively strong magnetic field, the valve magnet and the limit magnet rotate, and the force therebetween transforms into a repulsive force. Since the limit magnet is limited in transverse movement by the limit compartment, the valve magnet, under an effect of the repulsive force, opens the outlet of the drug storage compartment; then, under a vibration effect of a vibration motor, a drug is released at an accelerated rate through a drug release channel.

### CROSS-REFERENCE TO RELATED APPLICATION

This application claims the priority benefit of China application serial no. 202510108161.0, filed on January 23, 2025. The entirety of the above-mentioned patent application is hereby incorporated by reference herein and made a part of this specification.

### BACKGROUND

### Technical Field

The disclosure relates to a field of medical devices, and more particularly, to a magnetically controlled vibrating drug delivery capsule robot and system.

### Description of Related Art

Gastrointestinal diseases seriously endanger human health, and prevalence thereof has remained high for a long time. Excessive use of drugs may easily lead to dependence and poses a risk of side effects. In addition, although oral drug administration is economical and practical, macromolecular drugs such as nucleic acids and proteins have problems such as being easily degraded and poorly absorbed (affected by mucus barrier) in a gastrointestinal environment, resulting in extremely low drug utilization. For example, insulin needed daily by diabetic patients has a utilization rate of less than one percent when taken orally, leading to drug administration only through direct injection, which is often accompanied by problems such as pain and discomfort.

In recent years, studies have shown that introducing vibration (in vitro ultrasonic vibration, in vivo micro-vibration, etc.) during drug absorption may effectively improve oral drug delivery efficiency of the macromolecular drugs. An enhancement principle thereof lies in eliminating the mucus barrier and improving contact efficiency between the drug and the intestine through vibration. However, existing drug delivery methods still remain at a passive drug delivery level, and drug release mainly relies on pH-triggered control or natural dissolution methods, which cannot achieve targeted drug delivery at specific locations.

For this problem, CN 117731219 A discloses a magnetically controlled capsule robot, including a capsule housing and a cargo compartment disposed inside the capsule housing. A substance exchange channel, a magnetic lock disposed around the substance exchange channel, and a magnetic switch valve disposed in cooperation with the magnetic lock are disposed on a wall of the capsule housing. The magnetic lock and the magnetic switch valve in the magnetically controlled capsule robot are respectively a magnetized magnetic lock and magnetized magnetic switch valve, and the magnetization of the magnetic lock and the magnetic switch valve adopts any one of the following two methods: (i) a magnetization direction of the magnetic switch valve is diverging outward with the magnetic switch valve as a center, and a magnetization direction of the magnetic lock is perpendicular to a curved surface or plane where the magnetic lock is located and directed outward; (ii) the magnetization direction of the magnetic switch valve is converging inward with the magnetic switch valve as the center, and the magnetization direction of the magnetic lock is perpendicular to the curved surface or plane where the magnetic lock is located and directed inward.

Manipulation of the above-mentioned capsule robot based on a magnetic soft valve requires a relatively high frequency alternating magnetic field (typically with a magnetic field frequency and amplitude greater than 20Hz and 30mT), relying on coils and AC power to implement. However, generating such magnetic field in a large-scale space such as a human body requires very high power supply power (several tens of kHz or above), and the coil heating problem is serious under long-term working conditions, making it difficult to implement lightweight and wearable design for an entire system. In addition, such capsule robots have no built-in motor drive, and vibrate strength is usually small, which limits performance of functions thereof.

Therefore, there is an urgent need for a new technical solution that achieves targeted drug delivery at specific locations while avoiding the above technical problems.

### SUMMARY

In view of the defects of the prior art, the purpose of the disclosure lies in providing a magnetically controlled vibrating drug delivery capsule robot and system, aiming to solve the problem that the prior art may not achieve targeted drug delivery at a specific location while avoiding problems such as high power supply requirements, large coil temperature rise, and low vibration intensity.

The first aspect of the disclosure relates to a magnetically controlled vibrating drug delivery capsule robot. The magnetically controlled vibrating drug delivery capsule robot includes a housing, and a vibration motor, a magnetically controlled component, a drug storage compartment, a limit compartment, a valve magnet, a limit magnet, and a power supply fixed in the housing;

The magnetically controlled component has a default disconnected state to drive the vibration motor to start and stop in response to presence of an external magnetic field;

A drug release channel is disposed between the drug storage compartment and an outer wall of the housing;

The limit magnet is placed in the limit compartment, and the valve magnet is located in the drug storage compartment;

When there is no magnetic field, the valve magnet blocks an outlet of the drug storage compartment under attractive force of the limit magnet;

When a first magnetic field is applied, the valve magnet blocks the outlet of the drug storage compartment under the attractive force of the limit magnet, and the vibration motor drives a drug delivery capsule to vibrate for stirring;

When a second magnetic field is applied, both the limit magnet and the valve magnet rotate in an axial direction in response to the magnetic field, and the valve magnet is subject to axial translation under repulsive force of the limit magnet to open the outlet of the drug storage compartment, and the vibration motor drives the drug delivery capsule to vibrate for drug release;

Intensity of the first magnetic field is less than intensity of the second magnetic field, the intensity of the first magnetic field is greater than a triggering magnetic field of the magnetically controlled component, and the intensity of the second magnetic field is greater than a triggering magnetic field of a magnet.

In some embodiments, the limit magnet and the valve magnet are spherical or cylindrical magnets, and are subjected to saturation magnetization pretreatment.

In some embodiments, the drug release channel is a Y-shaped three-way structure, a main pipe is located in the axial direction, an outlet is disposed at the drug storage compartment, two branch pipes are located in a cross section, and an outlet is disposed at the outer wall of the housing.

In some embodiments, the drug release channel is a two-way structure, a main pipe is located in the axial direction, an outlet is disposed at the drug storage compartment, a branch pipe is located in a cross section, an outlet is disposed at the outer wall of the housing, and the outer wall of the housing is provided with a transverse channel.

In some embodiments, the magnetically controlled component is at least one of a normally open reed switch, a normally closed reed switch, a Hall switch, a bipolar Hall latch, or a series-parallel combination thereof.

In some embodiments, the vibration motor is located at an end of the housing away from the magnetically controlled component, and is offset from an axis of a capsule.

In some embodiments, the vibration motor is located directly below the limit magnet.

In some embodiments, the housing is provided with a thread on an outside to stir a released drug and break mucus barrier.

The second aspect of the disclosure relates to a magnetically controlled vibrating drug delivery capsule system includes the magnetically controlled vibrating drug delivery capsule robot according to any embodiment of the disclosure and an external magnetic field generation device; The external magnetic field generation device is used to provide a magnetic field to a vibrating drug delivery capsule robot.

In some embodiments, an external control terminal is further included;

The external control terminal is used for communication connection with the magnetically controlled vibrating drug delivery capsule robot, sending a control instruction to change a duty cycle of a power supply output current waveform, thereby changing a vibration frequency of the vibration motor.

It may be understood that beneficial effects of the above second aspect may refer to the related description in the above first aspect, which will not be repeated here.

Overall, compared with the prior art, the technical solutions conceived in the disclosure have the following beneficial effects:

The disclosure provides the magnetically controlled vibrating drug delivery capsule robot, which is internally provided with the power supply and the motor, and may achieve relatively strong vibration intensity; internally provided with the valve magnet and the limit magnet, and in a default state, an attractive force exists between the two, and the valve magnet, under an effect of the attractive force, blocks the drug storage compartment export; under an effect of an external relatively strong magnetic field, the two rotate, and the force between the two transforms into a repulsive force. Since the limit magnet is limited in transverse movement by the limit compartment, the valve magnet, under an effect of the repulsive force, opens the outlet of the drug storage compartment; then, under a vibration effect of the vibration motor, the drug is released at an accelerated rate through the drug release channel, and at the same time, mucus barrier is cleared, thereby improving an absorption rate of the drug. The disclosure may achieve multiple drug administrations and stirring as needed, and compared to drug capsules with magnetic soft valves, uses a non-alternating small magnetic field for triggering, reducing the requirements for the power supply, and avoiding a coil heating problem.

### BRIEF DESCRIPTION OF THE DRAWINGS

FIG. 1 is an exploded view of a structure of a magnetically controlled vibrating drug delivery capsule robot provided in an embodiment of the disclosure.

FIG. 2 is a schematic view of a related structure of drug storage and drug release provided in an embodiment of the disclosure.

FIG. 3 is a schematic view of a structure of a drug release channel provided in an embodiment of the disclosure.

FIG. 4 is a schematic view of a threaded structure disposed outside a housing provided in an embodiment of the disclosure.

FIG. 5A is a schematic view of an operation of applying an external magnetic field provided in an embodiment of the disclosure, corresponding to application of a relatively high magnetic field, and FIG. 5B is a schematic view of an operation of applying an external magnetic field provided in an embodiment of the disclosure, corresponding to application a relatively low magnetic field.

FIG. 6 is a schematic view of mechanics between a limit magnetic bead and a valve magnetic bead provided in the embodiment of the disclosure.

### DETAILED DESCRIPTION OF DISCLOSED EMBODIMENTS

In order for the purpose, technical solutions, and advantages of the disclosure to be more clearly understood, the disclosure is further described in detail below with reference to the accompanying drawings and embodiments. It should be understood that the specific embodiments described herein are only used to explain the disclosure and are not used to limit the disclosure.

The embodiments of the disclosure are described below with reference to the accompanying drawings in the embodiments of the disclosure.

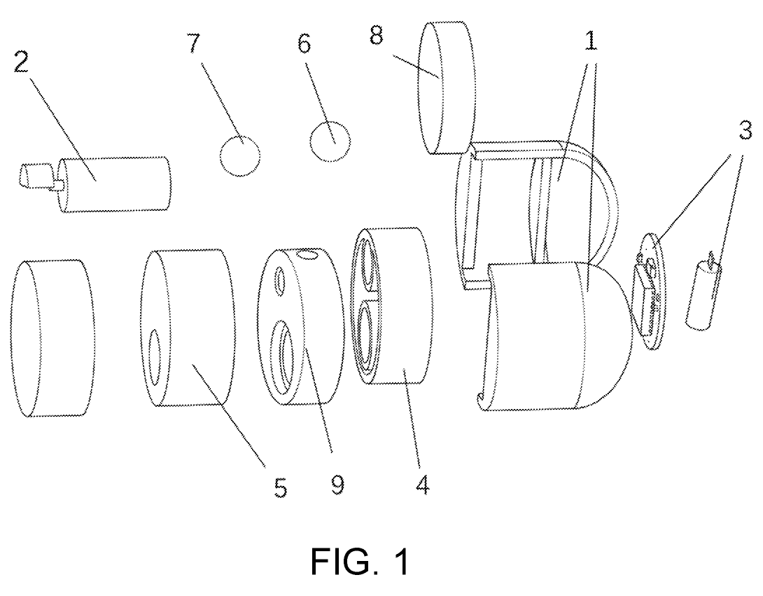

In the first aspect, as shown in FIG. 1, the disclosure discloses a magnetically controlled vibrating drug delivery capsule robot. The magnetically controlled vibrating drug delivery capsule robot includes a housing 1, a vibration motor 2 and a magnetically controlled component 3, a drug storage compartment 4, a limit compartment 5, a valve magnet 6, a limit magnet 7, and a power supply 8 fixed in the housing 1.

The magnetically controlled component 3 has a default disconnected state, and is used to drive the vibration motor to start and stop in response to presence of an external magnetic field.

A drug release channel 9 is disposed between the drug storage compartment 4 and an outer wall of the housing 1.

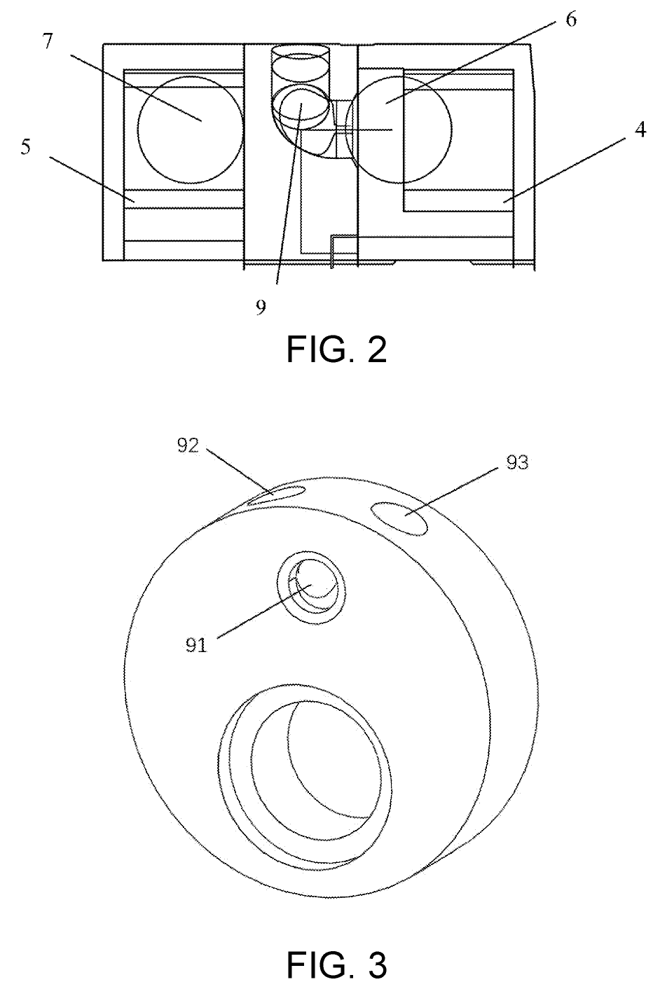

As shown in FIG. 2, the limit magnet 7 is placed in the limit compartment 5, and the valve magnet 6 is located in the drug storage compartment 4.

When there is no magnetic field, the valve magnet blocks an outlet of the drug storage compartment under attractive force of the limit magnet. When a first magnetic field is applied, the valve magnet blocks the outlet of the drug storage compartment under the attractive force of the limit magnet, and the vibration motor drives a drug delivery capsule to vibrate for stirring. When a second magnetic field is applied, both the limit magnet and the valve magnet rotate in an axial direction in response to the magnetic field, and the valve magnet is subject to axial translation under repulsive force of the limit magnet to open the outlet of the drug storage compartment, and the vibration motor drives the drug delivery capsule to vibrate for drug release. Intensity of the first magnetic field is less than intensity of the second magnetic field. The intensity of the first magnetic field is greater than a triggering magnetic field of the magnetically controlled component, and the intensity of the second magnetic field is greater than a triggering magnetic field of a magnet.

The magnetically controlled capsule includes a housing, and the housing is made of an impact-resistant material to ensure that the vibrating capsule will not damage the housing under vibrating conditions. The housing is also made of a biocompatible material that has passed safety standards. For example, pharmaceutical-grade polycarbonate is used as a material of the housing.

In this embodiment, the capsule may have any size or dimension provided that it may be placed inside a patient's body. Referring to FIG. 1, the capsule has a length from a front end to a rear end. In an embodiment, the length of the capsule is 28.2 mm. The capsule has a hemispherical end portion. A PCB board and a reed switch are placed inside the end portion. A diameter of the hemispherical end portion is a diameter of a capsule body. In an embodiment, a diameter of the capsule is approximately 12 mm, and a thickness of the housing is 0.65 mm.

In this embodiment, the capsule may have any weight and may be modified in weight according to a built-in battery, provided that movement or vibration does not cause obvious discomfort to the patient. In an embodiment, the weight of the capsule is less than 4 grams. In another embodiment, the weight of the capsule is less than 5 grams.

In an embodiment, the vibration motor 2 is an eccentric wheel motor. A motor and an eccentric wheel provide vibration power for the capsule, and by changing a duty cycle thereof, the vibration of the vibrating capsule has an adjustable frequency. The vibration motor may generate vibration with a frequency of 5 to 20Hz and a duty cycle adjustable from 0.1 to 1. The drug release in the disclosure is achieved through motor vibration, and a specific principle is flow rate difference, pressure difference, and inertia.

Vibration intensity of the vibrating capsule has a predetermined value, and the value is preset when the capsule leaves the factory. Each vibrating capsule may be customized to have different vibration intensity. In an implementation, a rotational speed of the vibration motor may be 100 revolutions per minute to 6000 revolutions per minute.

The limit magnet 7 is limited by the limit compartment 5, only supporting rotation in the axial direction, and may be not subject to transverse movement. The valve magnet 6 is only used for opening and closing the drug release channel 9. Magnitude of the triggering magnetic field depends on a width of the drug release channel and residual magnetization intensity of the spherical magnet. In this implementation scheme, the drug release channel is set to a diameter of 3mm, residual magnetic flux density of the spherical magnet is 1.21T, and the triggering magnetic field should be greater than 15mT.

In some embodiments, the limit magnet 7 and the valve magnet 6 are spherical or cylindrical magnets, and are subjected to saturation magnetization pretreatment.

It should be noted that in the disclosure, spherical or cylindrical magnets are preferably used, which are easier to achieve flipping movement.

In an embodiment, the limit magnet and the valve magnet are both spherical magnets. Residual magnetization intensity thereof is approximately 1.21T, and a model is a N35 neodymium iron boron magnet with the same magnetization method, and a diameter is slightly greater than a diameter of the drug release channel.

In some embodiments, as shown in FIG. 3, the drug release channel 9 is a Y-shaped three-way structure, the main pipe 91 is located in the axial direction, the outlet is disposed at the drug storage compartment, and the two branch pipes 92 and 93 are located in a cross section, and the outlet is disposed at the outer wall of the housing.

It should be noted that in the disclosure, the outlet of the drug release channel is preferably configured as two branch pipes. Since a liquid contact area is increased, liquid surface tension may be weakened to increase a drug release rate. The two branch pipes are located in the same plane, further reducing a space of the capsule.

In some embodiments, the drug release channel is a two-way structure, the main pipe is located in the axial direction, the outlet is disposed at the drug storage compartment, the branch pipe is located in the cross section, the outlet is disposed at the outer wall of the housing, and the outer wall of the housing is provided with a transverse channel.

It should be noted that in the disclosure, the transverse channel is preferably provided on the outer wall of the housing, which may weaken the liquid surface tension and increase the drug release rate due to the increased liquid contact area.

The magnetically controlled component may perform operations according to the external magnetic field, serving as a switch of the overall circuit. A basic principle of a triggering operation thereof is that when the triggering magnetic field is reached, the magnetically controlled component will close the overall circuit. In an embodiment, the triggering magnetic field of the motor is greater than 2mT. In another embodiment, the triggering magnetic field is less than 3mT. In another embodiment, the triggering magnetic field is less than 5mT.

In some embodiments, the magnetically controlled component is at least one of a normally open reed switch, a normally closed reed switch, a Hall switch, a bipolar Hall latch, or a series-parallel combination thereof.

In some embodiments, the vibration motor is located at an end of the housing away from the magnetically controlled component, and is offset from an axis of the capsule.

It should be noted that in the disclosure, a placement position of the motor is designed as described above. At this time, a center of gravity of the vibrating capsule is offset from the axis, transverse vibration is greater under an effect of the magnetic field, reducing self-rotation and enhancing a drug release/stirring effect.

In some embodiments, the vibration motor is located directly below the limit magnet.

It should be noted that a pair of magnets of the vibration motor itself may affect manipulation of a magnetic ball. In the disclosure, the motor is preferably placed directly below the limit magnet, which not only may avoid this effect, but also may make the structure more compact.

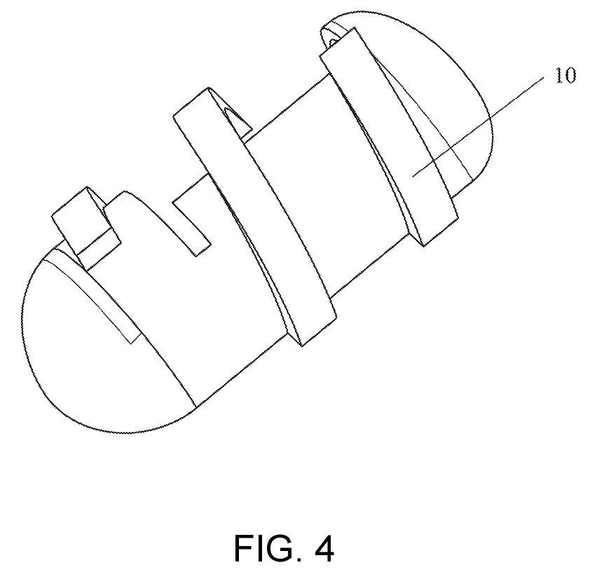

In some embodiments, as shown in FIG. 4, the housing is provided with a thread 10 on the outside to stir the released drug and break mucus barrier.

It should be noted that the thread on the outside of the housing may stir the released drug and break the mucus barrier, thereby increasing drug absorption efficiency.

In the second aspect, the disclosure discloses a magnetically controlled vibrating drug delivery capsule system, including the magnetically controlled vibrating drug delivery capsule robot according to any embodiment of the disclosure and an external magnetic field generation device;

The external magnetic field generation device is used to provide a magnetic field to a vibrating drug delivery capsule robot.

When the external magnetic field generation device is a permanent magnet, magnitude of intensity of the magnetic field is changed by changing a distance from the vibrating capsule thereof. When the external magnetic field generation device is a coil, the magnitude of intensity of the magnetic field is changed by changing a current thereof.

In an embodiment, the intensity of the first magnetic field is 3mT, and the intensity of the second magnetic field is 15mT.

In some embodiments, an external control terminal is further included;

The external control terminal is used for communication connection with the magnetically controlled vibrating drug delivery capsule robot, sending a control instruction to change a duty cycle of a power supply output current waveform, thereby changing a vibration frequency of the vibration motor.

The magnetically controlled vibrating drug delivery capsule robot, in addition to having a drug delivery function and promoting drug absorption, may also be used to provide massage by vibrating in a gastrointestinal tract.

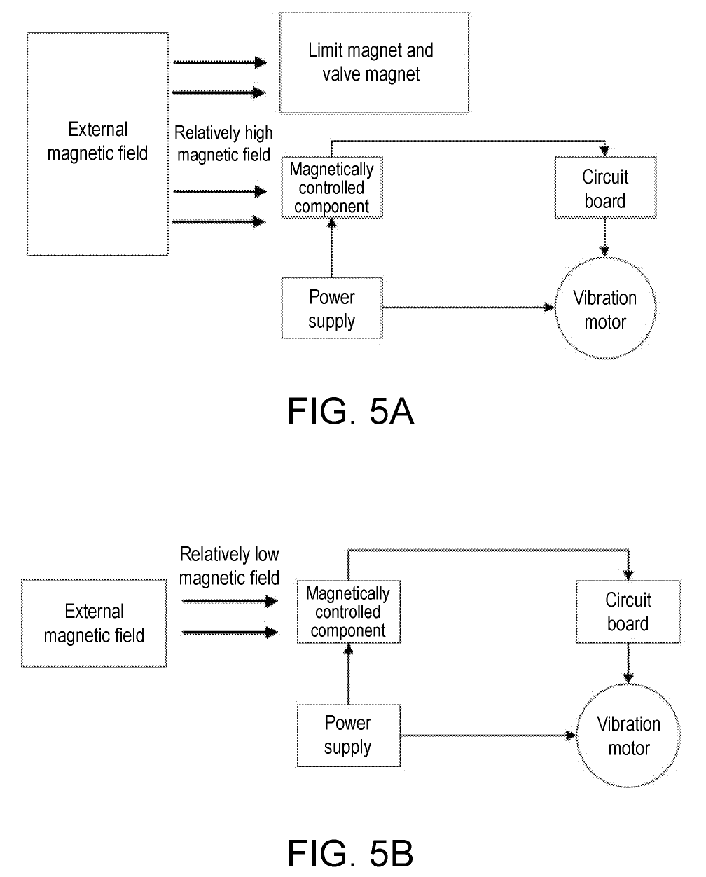

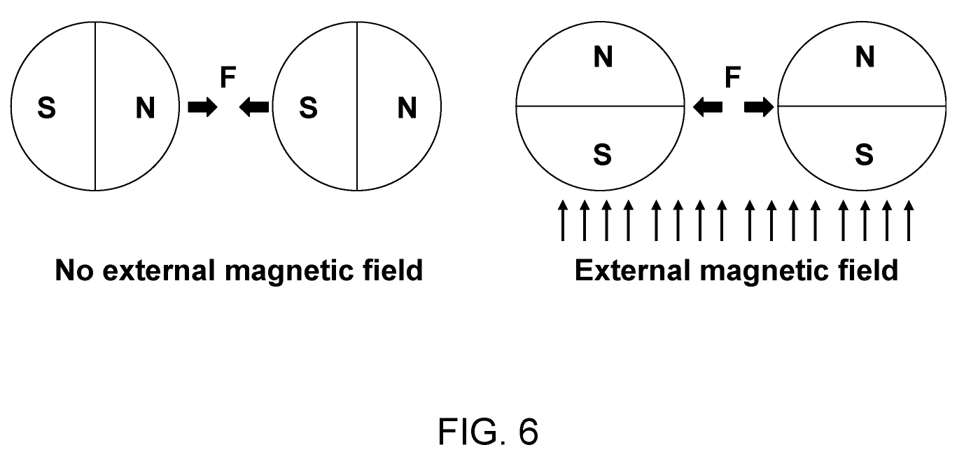

A complete working process of the magnetically controlled vibrating drug delivery capsule system involved in the disclosure is as follows: (1) during storage or before use of the capsule, the capsule is in a magnetic field-free environment, the entire circuit is disconnected, the vibration motor does not vibrate, the attractive force exists between the valve magnet and the limit magnet, and the valve magnet closes the outlet of the drug storage compartment; (2) the capsule is swallowed, and at this time, the capsule is in a free state; (3) the permanent magnet is placed at an outer position of a stomach, initially at a relatively small distance, i.e., a relatively high magnetic field, as shown in FIG. 5A, both the limit magnet and the valve magnet rotate in the axial direction in response to the magnetic field, as shown in FIG. 6, the valve magnet is subject to the axial translation under the repulsive force of the limit magnet, opening the outlet of the drug storage compartment, and the vibration motor drives the drug delivery capsule to vibrate for a round of drug release; (4) when it is necessary to stop the drug release midway and stirring is required, the distance between the permanent magnet and the stomach is increased, i.e., a relatively low magnetic field, as shown in FIG. 5B, the limit magnet and the valve magnet rotate back to an initial state, the valve magnet blocks the outlet of the drug storage compartment under the attractive force of the limit magnet, and the vibration motor drives the drug delivery capsule to vibrate for a round of stirring; (5) when it is necessary to stop the drug release midway and stop stirring, the distance between the permanent magnet and the stomach is further increased, at this time, a magnetically controlled switch is turned off, and the vibration motor is turned off, achieving a power-saving mode; (6) if a new round of drug release is required, step (3) is performed, and if a new round of stirring is required, step (4) is performed; (7) when the capsule runs out of power or is no longer needed for use, an external magnet is removed, and the capsule is discharged through a digestive tract.

In the disclosure, by controlling the magnitude of the magnetic field, the triggering magnetic fields of the magnet and the reed switch are different. Therefore, in response to the magnetic fields of different magnitude, corresponding operations are generated, thereby achieving decoupled control of vibration and drug delivery, controlling the dosage of drug release while increasing the drug absorption efficiency.

It should be understood that expressions such as "include" and "may include" that may be used in the disclosure indicate the existence of the disclosed functions, operations, or constituent elements, and do not limit one or more additional functions, operations, and constituent elements. In the disclosure, terms such as "include" and/or "have" may be interpreted as indicating specific features, numbers, operations, constituent elements, components, or combinations thereof, but may not be interpreted as excluding the existence or addition possibility of one or more other features, numbers, operations, constituent elements, components, or combinations thereof.

In addition, in the disclosure, the expression "and/or" includes any and all combinations of the associated listed words. For example, the expression "A and/or B" may include A, may include B, or may include both A and B.

In the description of the embodiments of the disclosure, it should be noted that, unless otherwise explicitly specified and limited, the term "connect" should be understood in a broad sense. For example, "connect" may be a detachable connection or a non-detachable connection; it may be a direct connection or an indirect connection through an intermediate medium. "Fixed connection" refers to being connected to each other, and a relative positional relationship remains unchanged after connection. "Rotational connection" refers to being connected to each other, and being capable of rotating relative to each other after connection. "Sliding connection" refers to being connected to each other, and being capable of sliding relative to each other after connection. The directional terms mentioned in the embodiments of the disclosure, such as "top", "bottom", "inner", "outer", "left", "right", etc., are only for reference to the directions of the accompanying drawings. Therefore, the directional terms used are for better and clearer description and understanding of the embodiments of the disclosure, rather than indicating or implying that the referred device or element must have a specific orientation, be constructed and operated in a specific orientation. Therefore, they should not be understood as limitations on the embodiments of the disclosure.

In addition, in the embodiments of the disclosure, the mathematical concepts mentioned, such as symmetry, equality, parallelism, perpendicularity, etc., are all limitations relative to current technological levels, not absolute and strict mathematical definitions, and a small amount of deviation is allowed, approximately symmetrical, approximately equal, approximately parallel, approximately perpendicular, etc. are all acceptable. For example, A being parallel to B means that A and B are parallel or approximately parallel, and an angle between A and B may be between 0 degrees and 10 degrees. A being perpendicular to B means that A and B are perpendicular or approximately perpendicular, and the angle between A and B may be between 80 degrees and 100 degrees.

The above descriptions are only specific implementation methods of the disclosure, but the protection scope of the disclosure is not limited thereto. Any changes or substitutions that may be easily conceived by those skilled in the art within the technical scope disclosed in the disclosure should be covered within the protection scope of the disclosure. Therefore, the protection scope of the disclosure should be subject to the protection scope of the claims.

What is claimed is:

1. A magnetically controlled vibrating drug delivery capsule robot, comprising a housing, and a vibration motor, a magnetically controlled component, a drug storage compartment, a limit compartment, a valve magnet, a limit magnet, and a power supply that are fixed in the housing;
wherein the magnetically controlled component has a default disconnected state to drive the vibration motor to start and stop in response to presence of an external magnetic field;
a drug release channel is disposed between the drug storage compartment and an outer wall of the housing;
the limit magnet is placed in the limit compartment, and the valve magnet is located in the drug storage compartment;
when there is no magnetic field, the valve magnet blocks an outlet of the drug storage compartment under attractive force of the limit magnet;
when a first magnetic field is applied, the valve magnet blocks the outlet of the drug storage compartment under the attractive force of the limit magnet, and the vibration motor drives a drug delivery capsule to vibrate for stirring;
when a second magnetic field is applied, both the limit magnet and the valve magnet rotate in an axial direction in response to the magnetic field, and the valve magnet is subject to axial translation under repulsive force of the limit magnet to open the outlet of the drug storage compartment, and the vibration motor drives the drug delivery capsule to vibrate for drug release;
intensity of the first magnetic field is less than intensity of the second magnetic field, the intensity of the first magnetic field is greater than a triggering magnetic field of the magnetically controlled component, and the intensity of the second magnetic field is greater than a triggering magnetic field of a magnet.

2. The magnetically controlled vibrating drug delivery capsule robot according to claim 1, wherein the limit magnet and the valve magnet are spherical or cylindrical magnets, and are subjected to saturation magnetization pretreatment.

3. The magnetically controlled vibrating drug delivery capsule robot according to claim 1, wherein the drug release channel is a Y-shaped three-way structure, a main pipe is located in the axial direction, an outlet is disposed at the drug storage compartment, two branch pipes are located in a cross section, and an outlet is disposed at the outer wall of the housing.

4. The magnetically controlled vibrating drug delivery capsule robot according to claim 1, wherein the drug release channel is a two-way structure, a main pipe is located in the axial direction, an outlet is disposed at the drug storage compartment, a branch pipe is located in a cross section, an outlet is disposed at the outer wall of the housing, and the outer wall of the housing is provided with a transverse channel.

5. The magnetically controlled vibrating drug delivery capsule robot according to claim 1, wherein the magnetically controlled component is at least one of a normally open reed switch, a normally closed reed switch, a Hall switch, a bipolar Hall latch, or a series-parallel combination thereof.

6. The magnetically controlled vibrating drug delivery capsule robot according to claim 1, wherein the vibration motor is located at an end of the housing away from the magnetically controlled component, and is offset from an axis of a capsule.

7. The magnetically controlled vibrating drug delivery capsule robot according to claim 6, wherein the vibration motor is located directly below the limit magnet.

8. The magnetically controlled vibrating drug delivery capsule robot according to claim 1, wherein the housing is provided with a thread on an outside to stir a released drug and break mucus barrier.

9. A magnetically controlled vibrating drug delivery capsule system, comprising the magnetically controlled vibrating drug delivery capsule robot according to claim 1 and an external magnetic field generation device;
wherein the external magnetic field generation device is configured to provide a magnetic field to a vibrating drug delivery capsule robot.

10. The magnetically controlled vibrating drug delivery capsule robot according to claim 9, wherein, further comprising: an external control terminal;
the external control terminal is configured for communication connection with the magnetically controlled vibrating drug delivery capsule robot, sending a control instruction to change a duty cycle of a power supply output current waveform, thereby changing a vibration frequency of the vibration motor.
